(19) United States
(12) Patent Application Publication
(10) Pub. No.: US 2025/0281663 A1
(43) Pub. Date: Sep. 11, 2025

(54) WOUND CONTACT LAYER WITH INFECTION-INHIBITING PROPERTIES

(71) Applicant: PAUL HARTMANN AG, Heidenheim (DE)

(72) Inventors: Markus Kettel, Heidenheim (DE); Jonathan Nosworthy, Neu-Ulm (DE); Stephen Law, Liverpool (GB); Steven Percival, Liverpool (GB); Rui Chen, Liverpool (GB)

(21) Appl. No.: 19/072,579

(22) Filed: Mar. 6, 2025

(30) Foreign Application Priority Data
Mar. 7, 2024 (DE) 102024106518.4

(51) Int. Cl. A61L 15/44; A61F 13/00; A61L 15/18; A61L 15/20; A61L 15/26

(52) U.S. Cl. CPC A61L 15/44; A61F 13/00063; A61L 15/18; A61L 15/20; A61L 15/26; A61L 2300/102; A61L 2300/104; A61L 2300/214; A61L 2300/404; A61L 2300/606

(57) ABSTRACT

The present invention relates to a wound contact layer which is suitable for wound care. The wound contact layer comprises a mesh-like substrate which is partially or completely coated with an emulsion, wherein the emulsion comprises an antimicrobial composition containing a) ionic silver and/or silver nitrate, b) ionic zinc and/or zinc nitrate, and c) EDTA. The wound contact layer has an antimicrobial effect and is effective against biofilms. The wound contact layer enables wound care and promotes wound healing.

### CROSS-REFERENCE TO RELATED APPLICATIONS

The instant application claims priority to German patent application no. 10 2024 106518.4, filed Mar. 7, 2024, which is incorporated by reference herein to the extent that there is no inconsistency with the present disclosure.

### TECHNICAL FIELD OF THE INVENTION

The present invention relates to a wound contact layer comprising a mesh-like substrate which has two sides. The first side of the substrate is such that this side can be applied to a wound. At least on this first side there is an emulsion containing an antimicrobial composition. As active ingredients, this composition contains both silver ions and zinc ions and EDTA. The wound contact layer is particularly suitable for covering infected wounds. The ointment firstly gives the wound contact layer atraumatic properties, and secondly it ensures long-term and uniform release of the active ingredients present to the wound.

### BACKGROUND OF THE INVENTION

Chronic wounds still pose a problem in modern medicine. Especially in older people and risk patients, such as diabetics, there is an increased risk of injuries not healing completely or at all. In such a case, the wound healing process is disturbed for various reasons and there is a persistent defect in the skin barrier. This is firstly accompanied by a reduction in the quality of life for those affected. Secondly, the risk of an infection continues to increase with ongoing duration of tissue exposure. If such an infection occurs, the prognosis deteriorates further. Since the body's own metabolic processes in the area of the chronic wound are impaired and a regular immune response does not occur completely or at all, this can result in the pathogens multiplying at the infection site. As this progresses, a biofilm often forms, in which bacterial pathogens combine to form a community that exhibits increased resistance to biocides and antibiotics and therefore makes further treatment extremely more difficult. This stage increases the likelihood of further complications such as necrosis or sepsis.

Wound dressings with antibiotic efficacy are known from the prior art. However, these either are insufficiently effective against biofilms, have no wound-healing effect or exhibit traumatic properties when changing the dressing since they bond to wound constituents.

EP 1 755 569 B9 describes a wound dressing with ointment that additionally contains an antibacterial metal such as silver. However, this document instructs those skilled in the art to use an anhydrous or water-binding ointment in order to avoid releasing the antibacterial metal too quickly and too excessively. This means that the wound dressing is not suitable for the treatment of necrotic wounds or of wounds that are partially permeated with necroses. In addition, the antibacterial effect of such wound dressings is insufficient in the case of wounds that have biofilms.

Consequently, there is a need for a means for covering wounds that exhibits sufficient antimicrobial efficacy against biofilms, moisturises the wound, promotes the healing of chronic wounds, and additionally can be removed atraumatically and painlessly. In order to meet the demands of everyday clinical practice, the means of choice should additionally be immediately usable, have sufficient stability in the case of prolonged storage and be resistant to possible temperature fluctuations.

### SUMMARY OF THE INVENTION

The preceding object is achieved by providing a wound contact layer comprising a mesh-like substrate which is partially or completely coated with an emulsion, wherein the emulsion comprises an antimicrobial composition containing
a) ionic silver and/or silver nitrate,
b) ionic zinc and/or zinc nitrate and/or zinc sulfate, and
c) EDTA.

The wound contact layer according to the invention has excellent atraumatic properties, i.e. it does not bond to the wound or wound constituents. It is neither possible for tissue to grow into the wound contact layer nor for the wound contact layer to adhere to the wound bed. The emulsion present stimulates wound closure and protects the wound edges from softening (maceration). Lastly, the antimicrobial composition present is effective not only against human-pathogenic bacteria, but also against biofilms. In addition, after being applied to the patient, the wound contact layer can be overlaid and fixed by a suitable secondary dressing. The type of secondary dressing may be specifically selected here depending on the wound type in order to ensure the best possible care.

The wound contact layer according to the invention may also be used if there are antibiotic-resistant bacteria in the wound to be treated. While antibiotics are usually organic compounds that are for example broken down by resistant bacteria, such an occurrence of resistances is not to be expected with the antimicrobial composition of the present invention.

The wound contact layer according to the invention can have properties that promote wound healing, even in the case of non-infected wounds.

The text below explains how the wound contact layer can be structurally and chemically formed in order to provide the greatest possible benefit in practice.

### BRIEF DESCRIPTION OF THE DRAWINGS

The figures are explained in more detail below. Where microbe number reductions are shown, they are based on a decadic-logarithmic scale of the ordinate:

FIG. 1a shows the release rate of the antimicrobial composition from an emulsion with about 1.4% by weight of this composition (based on the ionic forms) to simulated wound fluid over a total period of 72 h. The antimicrobial composition in the emulsion consisted of 18% by weight of tetrasodium EDTA, 39% by weight of silver nitrate and 43% by weight of zinc nitrate.

FIG. 1b shows the release rate of the antimicrobial composition from an emulsion with 1.94% by weight of this composition (based on the ionic forms) to simulated wound fluid over a total period of 72 h. The antimicrobial composition in the emulsion consisted of 18% by weight of tetrasodium EDTA, 39% by weight of silver nitrate and 43% by weight of zinc nitrate.

FIG. 2 shows the reduction of biofilms, produced in a CDC bioreactor, of P. aeruginosa (solid bars) and S. aureus (hatched bars) by wound contact layers according to the invention depending on the water concentration of the emulsion at an active ingredient content of the antimicrobial composition in the emulsion of about 0.94% by weight (based on the ionic forms). The water concentration of the emulsion (7.5%, 12% and 24% by weight) is plotted on the abscissa. Here, solid bars represent the strain P. aeruginosa (ATCC 15442) and hatched bars represent S. aureus (ATCC 6538).

FIG. 3 shows the reduction of biofilms, produced by means of drip flow bioreactor, of various organisms by wound contact layers according to the invention with about 1.94% by weight of the antimicrobial composition (based on the ionic forms) in the emulsion. The antimicrobial composition in the emulsion consisted of 18% by weight of tetrasodium EDTA, 39% by weight of silver nitrate and 43% by weight of zinc nitrate.

FIG. 4 shows the comparison of efficacy between the emulsion specified for FIG. 3 (solid bars) and an emulsion that likewise contained about 1.94% by weight of the antimicrobial composition (based on the ionic forms), but in this case consisting of 86% by weight of tetrasodium EDTA, 6.5% by weight of silver nitrate and 7.5% by weight of zinc nitrate (hatched bars). The comparison is based on a selection of certain organisms from FIG. 3.

FIG. 5 shows the reduction in the cell count of pathogenic organisms in a contact test in accordance with Standard AATCC 100. Tested were wound contact layers according to the invention with about 1.94% by weight of antimicrobial composition in the emulsion (based on the ionic forms). The antimicrobial composition consisted of 18% by weight of tetrasodium EDTA, 39% by weight of silver nitrate and 43% by weight of zinc nitrate. The wound contact layers were each tested in triplicate. The values shown are average values.

### DETAILED DESCRIPTION OF THE INVENTION

Within the meaning of the invention, the term “medically acceptable material” is a nontoxic, lint-free and stable substance that, under normal conditions, does not dissolve in either polar or nonpolar compounds and cannot be broken down or liquefied to any significant extent by the secretions of either animal or bacterial cells.

The term “stable emulsion” means that the polar and nonpolar constituents of an emulsion (for example water and oil) do not separate into two phases in a temperature range of 20° C. to 25° C. and at a pressure of 1 bar. If no separation occurs under these conditions within a period of seven days, it can be assumed that the emulsion is stable. [0027]“t-EDTA” refers to the compound tetrasodium ethylenediaminetetraacetic acid.

The term “biofilm” means a thin, two-dimensionally spread mucosal film in which there are populations of microorganisms. The mucosal film is formed by the microorganisms and is a matrix of extracellular polymeric material that includes the microorganisms. Typically, the populations of microorganisms are mixed populations. Biofilms form on surfaces, which can also include wound tissue. The microorganisms organized in the biofilm exhibit elevated resistance to conventional antibiotics, to disinfectants and to the immune system of more advanced forms of life.

The term “biofilm-reducing” encompasses both the killing of bacteria in the biofilm (disinfecting properties) and the breaking of chemical bonds, and the removal of chemical binding partners from the biofilm-forming extracellular matrix. The latter generally lowers the defences of the microorganisms previously organized in the biofilm against effects such as disinfectants, antibiotics, the immune system or environmental influences. In the context of the present invention, “biofilm-reducing” is understood to mean only those substances that are suitable for application to open wounds without displaying any noticeable harmful effect on the individual to be treated.

The term “mesh-like substrate” means that the substrate is in the form of a mesh with webs and openings between the webs. In particular, there are openings that extend from one side of the mesh-like substrate to the opposite side. Within the meaning of the present invention, an opening should therefore be understood to mean a cavity that passes through the substrate and is suitable for allowing a liquid such as wound exudate to pass from one surface of the substrate to the opposite surface of the substrate.

Within the meaning of the present invention, a mesh-like structure may be produced for example by a grid, a woven fabric, a knitted fabric, a perforated nonwoven or by a perforated film layer.

The expression “colony-forming unit” (CFU) means a single division-capable cell of a unicellular organism, in particular of a human-pathogenic unicellular organism or bacterium.

“Coated” means that a surface of a solid is at least partially covered or overlaid by means of a substance that differs from the structure of the solid.

The expression “configured for application to a wound” means intended and suitable for application to a wound for a therapeutic purpose in the sense of wound treatment.

The term “atraumatic” means that a wound care product does not bond firmly to the wound, that is to say for example does not dry out in the wound or merge therewith, and that it is possible to painlessly remove the product without disturbing the healing process.

The present invention relates to a wound contact layer. This wound contact layer may be used alone or if required, after application to a wound, be overlaid by an absorbent secondary dressing or be attached to the site of the wound by means of secondary dressing, adhesive film, adhesive tape or other fixing agents. If required, for example if the secondary dressing is saturated with wound exudate or if the attached adhesive fastening detaches, the secondary dressing or adhesive can be replaced without having to detach the wound contact layer from the wound. The wound is left undisturbed in this way and undesirable drying-out of the secondary dressing in the wound is avoided.

It is advantageous that the wound contact layer adheres atraumatically to the skin by means of the emulsion present. An additional secondary dressing offers the advantage of preventing shifting of the wound contact layer. In most cases, the wound contact layer does not have to be held at the wound site until a fixing agent is applied, but initially holds on its own, and so the user has both hands available for the preparation of the secondary dressing.

The wound contact layer according to the invention is suitable for acute and bleeding wounds, chronic wounds, exuding wounds, burns (up to second-degree burns), wounds containing necrotic tissue or fibrin coatings, especially for infected wounds and among these in particular biofilm-containing wounds. Mixed forms of these wounds often occur in practice, for example infected, partially necrotic, chronic wounds with biofilm. The advantageous properties of the wound contact layer according to the invention are particularly evident in such cases, since a variety of different pathological wound environments are treated with only one product.

The wound contact layer comprises at least one mesh-like substrate and an emulsion which contains an antimicrobial composition. Further constituents, such as a peripheral adhesive edge, may be added if required in order to adapt the wound contact layer to the intended purpose. This is explained in more detail elsewhere.

In the context of the present invention, provision may be made for the emulsion to exclusively be on the first side of the substrate and thus on that side which faces the wound. In this way, the side intended for wound contact can clearly be recognized by the user and does not require further identification. Moreover, this embodiment has the economic advantage that emulsion material is saved without compromising the wound care.

The substrate present in the wound contact layer according to the invention is a flexible and elastic solid that can adapt to the shape of the body or wound surface. The substrate consists of a solid, insoluble, medically acceptable material or of fibres produced therefrom, where the material preferably has a crystal lattice structure and is not amorphous. The material may be a polymer or a polymer mixture. Preferably, the substrate is flat or planar, with the result that it has a substantially uniform height and neither the first nor the second side have elevations. Minor production-related tolerances can be neglected here, and so within the meaning of the invention a uniform height should be considered to be given even in the case of deviations of +/−5%. A flat substrate may have a rectangular, square, oval or round shape in plan view, with oval or round shapes being particularly suitable for wounds on joints and rectangular or square shapes being able to be packaged more easily and using storage space more efficiently. The wound contact layer may have the shape of the substrate, however this is not absolutely necessary.

The substrate has a first side which faces the wound in use (proximal orientation) and a second side which is on the opposite side to the first side and faces away from the wound in use (distal orientation).

The substrate is configured to be mesh-like. The mesh-like configuration allows the passage of wound exudate starting from the first side of the substrate in the direction of the second side of the substrate. In addition, the mesh shape gives the substrate pronounced flexibility, since the material stress when applied to a wound and the associated (reversible) deformation is minimized. Last but not least, the mesh reduces the contact surface with the wound and thus the interfacial tension, with the result that the substrate can be detached from the wound in a particularly gentle manner.

One possible mesh shape is the shape of a grid, in which the substrate contains webs in the form of longitudinal and transverse struts which are arranged essentially perpendicular to one another, with the result that the openings in the substrate take the shape of rectangles. According to a preferred embodiment, the mesh-like configuration is in the form of a uniform pattern, with the result that all openings or pores have essentially the same spatial extension and the same distance to one another. The advantage that results from this is that essentially constant parameters and forces prevail over the entire surface of the first and second side and the antimicrobial composition can therefore act uniformly at all positions of a wound.

Preferably, the substrate contains fibres. The substrate may exclusively consist of fibres. These fibres may contain or consist of one or more of the following materials: polyamide, polyester, polyacrylic, polyurethane, polypropylene, polyvinyl alcohol, cotton, viscose and mixtures thereof. An example of a possible mixture of fibres of various materials is a combination of polyester with cotton or of polyester with viscose. When using polyamide as fibre material, the polyamide may be in the form of nylon.

All of the fibres listed may be configured as knitted fabric for use in the mesh-like substrate. It is also possible to arrange the fibres as tulle. A mesh in the form of a tulle may be characterized by a repetitive arrangement of hexagons (hexagonal arrangement within the tulle) or rhombuses (rhombus arrangement within the tulle).

In the present case, as a mesh-like substrate, all fibres or types of fibres listed above firstly have structuring properties and in addition the advantageous property of transferring fluids such as wound exudate to an optional secondary dressing, where they can be stored and later removed. In combination with a water-containing emulsion, the wound is additionally rinsed to a small extent, thus promoting the detachment of any necroses present. In general, fibres of natural origin are more hydrophilic here than synthetic fibres. However, synthetic fibres outperform natural fibres in terms of their binding affinity to nonpolar substances such as oils, fats and waxes. For this reason, the synthetic fibres have excellent binding properties with respect to W/O emulsions, which may be used to coat the wound contact layer according to the invention. In addition, they are inexpensive, tear-resistant, have high tensile strength and are easy to process. Due to their nonpolar character, they do not exhibit any retention behaviour towards the cations of the antimicrobial composition, resulting in their optimal release.

Natural fibres offer the advantage of a better life-cycle assessment and form particularly stable coatings with O/W emulsions. However, when using natural fibres, it may be necessary to increase the concentration of antimicrobial composition or to coat them with a larger amount of emulsion in order to obtain the same efficacy as with synthetic fibres, since natural fibres, on account of their polar properties, can interact with these charged particles and inhibit their release to a small extent.

In addition, it is also possible that the mesh-like substrate does not consist of fibres, but of an in particular homogeneous mass. The mass should be elastic, flexible, resistant and naturally biocompatible. A suitable mass is silicone, for which reason the mesh-like substrate may consist of or contain silicone in the context of the invention. Silicone offers the advantage of the greatest possible biocompatibility. It is hypoallergenic and has no toxicity whatsoever. Furthermore, it is also biochemically inert. It cannot be decomposed by microorganisms or pathogens or used as nutrient substrate, does not bond to the wound tissue and can be removed painlessly. It is often used in the medical field for these reasons. Nevertheless, silicone has a disadvantage. It is known for its occlusive property, which hinders skin respiration and inhibits absorption of wound exudate into wound dressings. Since, however, the support of the wound contact layer according to the invention has openings due to its mesh-like structure, the desired mass transfer is retained.

In the context of the invention, it is indeed possible in principle to form a film as a mesh-like substrate. In the case of dry or scab-covered wounds, film-containing mesh-like substrates may be an alternative to other substrate forms, even though the emulsion in the case of a film (as opposed to a fibre-based substrate) can be applied only to the surfaces of the films and not additionally within the substrate. Preferably, the mesh-like substrate comprised by the wound contact layer is therefore not a film and also does not contain a film.

In the context of the invention, the substrate may, if required, be free of certain substances. For example, the substrate may be free of gelatine and collagen or generally be free of substances of animal origin or of substances of natural origin. Substrates that are free of substances of this kind are generally resistant to undesirable biodegradation—for example when treating an infected wound.

According to one embodiment, the mesh-like substrate is coated with elemental, i.e. nonionic, silver. This additional silver, which is present in addition to the ionic silver present in the antimicrobial composition, serves as an additional reservoir. The fact that it is not present in the emulsion but rather is covered by it enables particularly long-term release of silver to the wound site. It is the case here that the more emulsion that has been coated on the substrate and the more hydrophobic the emulsion, the more delayed the additional silver is released from the substrate.

The mesh-like construction means that openings are present in the substrate, although distances between individual fibres or fibre filaments such as those found in every textile composite (knitted fabric, tulle, etc.) are not considered to be openings within the meaning of the invention.

The openings present in the mesh-like substrate may be of any desired shape. A circular or elliptical configuration is possible, since this avoids corners which can feel uncomfortable for the patient after a prolonged period of wear on a wound. Alternatively, a square or trigonal opening area is likewise possible. The size of the openings may vary in the context of the present invention, but these should be large enough to enable or to not slow the outflow of exudate. The openings may have an area of at least 0.01 mm² and preferably an area of at least 0.1 mm². Pores are not openings within the meaning of the invention. Openings having a projection area of at least 0.1 mm², corresponding to a circle diameter of 0.357 mm, are of particularly good suitability. On the other hand, with excessively large openings, there is the risk of adhesion to the wound and the growth of granulation tissue into the openings. Openings with a size of 0.1 mm² to 10 mm², of 0.3 mm² to 8 mm², particularly 0.5 to 5 mm², are of good suitability. It is also possible to use openings that have an opening area of at most 1 mm².

The substrate has a plurality of openings and a closed region (webs) surrounding the openings. The ratio of the total area of the openings to the closed area of the substrate is 1:1.5 to 1:4, preferably 1:2 to 1:3.

In the context of the present invention, an emulsion may be an ointment, a hydrophobic ointment, a hydrophilic ointment, a water-absorbing ointment, an oil-in-water emulsion (O/W emulsion), a water-in-oil emulsion (W/O emulsion), a gel or a paste.

The emulsion may be water-binding, fat-binding or both. It contains at least one polar and at least one nonpolar constituent.

The polar constituent may be water or another nontoxic polar substance that is preferably present as a liquid at room temperature. One possible example is PEG, which may be used as a liquid (molecular mass of below 600 g/mol) or as a powder (molecular mass from 2000 g/mol) or as a paste (molecular mass between 600 and 2000 g/mol) and is present dissolved in the finished emulsion.

The nonpolar constituent may for example be a substance that is one of oils, fats, fatty acids, waxes, triglycerides, and hydrocarbons. In the case of hydrocarbons, preference is given to aliphatic hydrocarbons. Particular preference is given to saturated, aliphatic hydrocarbons. However, the use of unsaturated hydrocarbons is possible as long as the resulting emulsion has a sufficient viscosity for it to produce a stable coating on the substrate. These nonpolar substances reduce the release of the components from the antimicrobial composition (barrier effect). This may be desirable if the wound contact layer is to remain on the wound for a relatively long time (for example several days) and continuously release the antimicrobial components.

In addition, mixtures of multiple polar and nonpolar substances are possible as a constituent of the emulsion.

Furthermore, the emulsion may contain one or more emulsifiers and water-binding constituents. Examples of water-binding constituents are propylene glycol, ethylene glycol, polyethylene glycol (PEG), in particular PEG 2000 (molecular weight 2 kDa) and mixtures of at least two of these constituents. These or other water-binding constituents may for example account for 1% to 20% by weight of the emulsion. A proportion of 5% to 15% by weight, particularly 8% to 12% by weight, is recommended. Alternatively, the proportion may also be at least 5% by weight. The water-binding constituents are also suitable for binding other polar liquids than water. Examples of other polar liquids are ethanol and glycerol. The water-binding constituents control the release of the antimicrobial composition. The higher their proportion and the proportion of polar liquid in the emulsion, the more components of the antimicrobial composition are released to the surroundings, in particular to the tissue, within a certain period. Examples of emulsifiers are stearates, cetearyl glucoside, lecithin and mixtures of at least two of these constituents. These and other emulsifiers may for example account for a proportion of 0.5% to 5% by weight in the emulsion.

A hydrophobic ointment is understood to mean an ointment having a base that does not contain any polar constituents or emulsifiers and is therefore not able to actively bind water. A hydrophobic ointment may contain a mixture of higher aliphatic alkanes as a base. For example, use may be made of petroleum jelly. More specifically, petroleum jelly is understood to mean a highly viscous substance containing both branched-chain solid hydrocarbons and straight-chain liquid hydrocarbons. A preferred variant of petroleum jelly is what is known as white petroleum jelly (Vaselinum album), which has a particularly high quality on account of its purity. The advantages of petroleum jelly include good biocompatibility and excellent skin tolerability. The occlusive properties and the almost non-existent absorption by the skin mean that petroleum jelly is well suited for providing longer-term protection in the case of defects in the skin barrier. Since it is almost impossible for microorganisms to metabolize petroleum jelly, pathogens cannot use it as a carbon source. Alternatively or additionally, a hydrophobic ointment may contain silicone oil. For example, the silicone oil may be present in the ointment in a concentration of 3% to 50% by weight or 5% to 30% by weight.

A water-absorbing ointment is understood to mean an ointment that contains not only lipophilic base materials but also amphiphilic auxiliaries and is therefore capable of absorbing water. Amphiphilic auxiliaries act as emulsifiers here. Suitable for water-absorbing ointments are W/O emulsifiers, preferably partial esters of glycerol or diglycerol esterified with various fatty acids, particularly preferably diglycerol partial esters with various medium- and long-chain fatty acids. One example of a suitable emulsifier is cetylstearyl alcohol. Water-absorbing ointments may contain lanolin (wool wax). For example, lanolin may be present in a concentration of 3% to 50% by weight or of 10% to 30% by weight. A preferred water-absorbing ointment contains 40% to 70% by weight of triglycerides, 15% to 30% by weight of diglycerol partial esters, 2% to 15% by weight of polyethylene glycol (PEG) and 5% to 25% by weight of water or another polar liquid. Preferably, the water-absorbing ointment contains 5% to 15% by weight of PEG, particularly preferably 5% to 12% by weight of PEG.

A hydrophilic ointment is understood to mean an ointment that is water-miscible. A hydrophilic ointment typically contains polyethylene glycols. Hydrophilic ointments assist the passage of wound exudate through the wound contact layer and simultaneously also the diffusion and release of the ions from the antibacterial composition.

An emulsion is understood to mean an ointment that contains an aqueous phase in addition to a lipid phase. Both W/O-type emulsions and O/W-type emulsions may be used in the context of the invention. Also possible are distributions in which two or more adjacent phases are formed.

A gel is understood to mean a preparation in which liquids are solidified by gel scaffold formers. Gels preferred for wound care are hydrophilic water-containing gels, which are also referred to as hydrogels. In addition to water (what are known as hydrogels) or another polar biocompatible liquid, suitable gels for example contain at least one scaffold former or gel former such as modified or unmodified polysaccharides, in particular hydroxymethyl cellulose, carboxymethyl cellulose, starch, modified starch, chitosan, alginate or agar, and gelatine, collagen, polyvinyl alcohol, polyglycidol, or polyacrylamide. These constituents or a mixture thereof may account for 0.5% to 30% by weight of the emulsion. In the context of the invention, a gel always contains one or more nonpolar constituents in addition to a polar liquid, where the proportion of the polar liquid in the gel is at least 50% by weight. Gels are particularly well suited for treating wounds in which there are fibrin coatings or necroses.

A paste is understood to mean an ointment with a solids content, where a solids dispersion is present. An ointment may for example contain a solids content of 0.1% to 15% by weight. Preferably, the solids content in the ointment is 0.3% to 5% by weight and particularly preferably 0.5% to 3% by weight. The antibacterial composition—if partially undissolved and present as a solid—is not taken into account in the specified solids contents. The optimal solids content depends on the type of solid or solids and whether they are present for example as an active ingredient or only as a skin care ingredient. A paste is usually a solids-stabilized ointment. The prerequisite is that the ointment contains water and that at least some of the solid particles present can form a hydrate shell, as is the case for example with inorganic ions.

Furthermore, the emulsion may also be present as a fluid. An emulsion containing the antimicrobial composition may also be present as a disperse fluid.

In order to adjust the consistency of the emulsion (for example if it is present as an ointment), the constituents that follow may be admixed. Each of these constituents may be present in a concentration of 1% to 10% by weight or 2% to 9% by weight or else 3% to 8% by weight: cetyl alcohol, stearyl alcohol, isopropyl myristate. It is pointed out that, after the wound contact layer has been applied to the tissue, nonpolar constituents lose viscosity due to body heat, which is entirely within the meaning of the invention and promotes the distribution of the emulsion in the wound area, with the result that there is no need for direct contact between substrate and wound bed in order to introduce the antimicrobial composition into the wound bed and to thereby also achieve an advantageous depth effect.

Finally, provision may be made for the emulsion to comprise at least one constituent selected from the group of mono-, di- or triglycerides, fatty acid esters, oligomers of glycerol, fatty alcohols, fatty acid esters, ethoxylated fatty alcohols, ethoxylated fatty acids, polyethoxylene derivatives or dimerized fatty acid esters or mixtures of the mentioned substances, where it is possible for these to be present in the emulsion for example in a content of 30% to 70% by weight or 40% to 70% by weight. Among these mentioned substances, preference is given to the following: di- and triglycerides of caprylic acid and/or of capric acid and/or of isostearic acid and/or of stearic acid and/or of 12-hydroxystearic acid, and lanolin. In particular, the emulsion may comprise a mixture of various mono-, di- and triglycerides and/or fatty acid esters, oligomers of glycerol and/or polyethylene glycols. Advantageous is a preparation comprising 1% to 70% by weight of mono-, di- and triglycerides, 1% to 70% by weight of fatty acid esters oligomers of glycerol, 1% to 20% by weight of polyethylene glycol and 1% to 10% by weight of water and antimicrobial composition.

Furthermore, the emulsion or the emulsion compositions described above may also contain a preservative or a stabilizer in an amount of 0.1% to 2% by weight. Examples of suitable preservatives are for example benzoic acid, sorbic acid or parabens. Examples of suitable stabilizers are ascorbyl palmitates and tocopherol. Since the emulsion-containing wound contact layer is usually sterilized before use, in most cases the use of preservatives can be dispensed with in order to keep the production outlay low. Preferably, provision may also be made for the emulsion to generally not contain any preservatives and/or stabilizers in order to reduce the probability of the occurrence of allergies and intolerance reactions as far as possible.

The content of polar liquids, particularly of water, in the emulsion may be from 1% to 50% by weight. In some cases (for example if the emulsion is present as a gel), more than 50% by weight of polar liquids may also be desirable and useful. Preferably, the emulsion contains 15% to 45% by weight of polar liquids, better 20% to 40% by weight of polar liquids and best of all the emulsion contains 25% to 35% by weight of polar liquids, since such a concentration enables particularly good release of the antimicrobial composition and at the same time ensures excellent stability of the emulsion. In addition, this range has proven to be particularly effective against the problematic pathogen S. aureus. It is possible for two or more different polar liquids to be present in the emulsion. Examples of possible combinations of polar liquids are water and ethanol or water and glycerol.

Preferably, the emulsion is a stable emulsion. Surprisingly, it has been found that the antimicrobial composition according to the invention has a stabilizing effect on the emulsion, resulting in a solids-stabilized emulsion. This is accompanied by beneficial effects. In addition to stabilizing the pH (even in the absence of any buffer systems), the solids-stabilized emulsion offers excellent long-term durability without separation of the aqueous and oily phases occurring. This long-term stability is a particularly desirable property for medical articles, since products of this kind are purchased by medical institutions as part of discounted bulk orders and must be stored until they are used (which is usually not foreseeable). During storage, the products may well be subject to seasonal temperature fluctuations. The solids-stabilized emulsion according to the invention offers improved resistance both in the case of prolonged storage periods and to temperature fluctuations. This is also beneficial to the sterilization process.

Various formulations of the emulsion may be used to impart the properties desired for the intended purpose. For example, the addition of triglycerides may be used to enable refatting and to increase the antimicrobial depth effect. A high proportion of polar liquids can promote the detachment of fibrin and necroses from the wound. The aim when selecting the ingredients and their concentration in the emulsion should be to close the wound without pathogens remaining in the tissue.

In the context of the present invention, the “antimicrobial composition” may be present in particular in the following states:
a) as a mixture of ionic silver and/or silver nitrate and ionic zinc and/or zinc nitrate and/or zinc sulfate and EDTA or tetrasodium EDTA,
b) as the complex compound Ag₂Zn(EDTA),
c) as a solution of said complex compound in a polar liquid such as water or another nontoxic, polar solvent, or
d) as a solution of the starting components silver nitrate, zinc sulfate or zinc sulfate monohydrate and tetrasodium EDTA in a polar liquid such as water or another nontoxic, polar solvent, with the result that said complex compound is formed.

Unless stated otherwise, the “antimicrobial composition” is primarily in the form of the mixture mentioned under a), specifically preferably in the ionic forms. Use may however also be made of other salts of silver and zinc as the nitrates thereof, as long as the reactants can form the complex compound specified under b). Not all cations of silver and zinc have to react here to form the complex. The formation of a chemical equilibrium in the sense of a dissociation with forward and reverse reactions does not constitute a disadvantage.

The antimicrobial composition has both biofilm-reducing and antimicrobial properties. In the release of antimicrobial components, the distribution along the concentration gradient in the direction of the wound may play a role, where the cations supply moisture to the wound by means of the hydrate shell adhering to them. A moist wound environment promotes the healing process.

The antimicrobial composition present in the emulsion contains at least the following components:
a) ionic silver and/or silver nitrate, and
b) ionic zinc and/or zinc nitrate and/or zinc sulfate, and
c) ethylenediaminetetraacetic acid (EDTA) and/or tetrasodium EDTA.

Preferably, the antimicrobial composition contains 5% to 50% by weight of the components listed under a), particularly preferably 10% to 45% by weight and very particularly preferably 20% to 40% by weight.

Furthermore, the antimicrobial composition preferably contains 3% to 45% by weight of the components listed under b), particularly preferably 5% to 40% by weight and very particularly preferably 9% to 35% by weight.

Finally, the antimicrobial composition preferably contains 20% to 90% by weight of the components listed under c), particularly preferably 25% to 60% by weight.

These concentration figures may relate either to the ionic forms or exclusively to the salts (for example silver nitrate and zinc nitrate). In the initial state, the zinc nitrate may be present as the hexahydrate Zn(NO₃)₂×6H₂O. In the initial state, tetrasodium EDTA may be present as the tetrahydrate.

Within the antimicrobial composition, the individual components may preferably take the following concentration ranges: EDTA may be present in a concentration of 15% to 90% by weight or else 25% to 80% by weight. Preferably, the concentration of EDTA is at least 15% or at least 25% by weight. Silver may be present in a concentration of 5% to 50% by weight or else 10% to 40% by weight. Preferably, the concentration of silver is at least 5% or at least 10% by weight. Zinc may be present in a concentration of 3% to 45% by weight or else 6% to 40% by weight. Preferably, the concentration of zinc is at least 3% or at least 6% by weight.

According to a preferred embodiment, the antimicrobial composition contains 5% to 45% by weight of one of the following components: ionic silver, silver nitrate, ionic zinc, zinc nitrate or zinc sulfate. Simultaneously or else independently thereof, the antimicrobial composition may contain 15% to 90% by weight of EDTA.

According to another preferred embodiment, the antimicrobial composition contains 35% to 45% by weight of silver nitrate, 20% to 40% by weight of zinc nitrate and 15% to 30% by weight of tetrasodium EDTA.

It is recommended to adjust the ratio of the components in the antimicrobial composition such that both silver and zinc (or the salts thereof, such as nitrates) are each present in excess in relation to EDTA. The term “excess” relates to the weight concentration, with this automatically also achieving a molar excess in terms of the amount-of-substance concentration.

It is recommended to first distribute the components of the antimicrobial composition in a polar, nontoxic liquid and then to mix this liquid with the remaining constituents of the emulsion. The use of water is recommended here; particularly recommended is deionized or distilled water, since it does not contain ions (such as Na⁺) that could react with the components of the antimicrobial composition. According to a preferred embodiment, the polar liquid with the antimicrobial composition contains free cations of silver and zinc. This may be achieved by both silver and zinc each being present in excess in relation to EDTA. For example, the sum total of the amounts of substance of silver and zinc may be at least three times the amount of substance of EDTA. This means that the free cations are ready for biochemical reactions. For example, it has been found that silver primarily exhibits an effect against pathogenic unicellular organisms. Zinc is able to activate immune cells. Through its function as cofactor in various transcription factors and enzymatic reactions, zinc promotes wound closure and also protects cells from apoptosis, which is induced by oxidative stress or bacterial toxins. By binding calcium, EDTA can decompose existing biofilms and likewise suppress their formation.

The release of the cations (Ag⁺ and Zn²⁺) from the wound contact layer according to the invention over the course of 24 h may be 1 to 200 μg per 100 cm² of substrate (in 100 ml of water, over 24 h, at 37° C.). The release is preferably 10 to 180 μg, better still 20 to 160 μg or 30 to 100 μg. The value may be adjusted by way of the composition of the emulsion, the proportions within the antimicrobial composition, the water content and the coating amount of the emulsion on the substrate. In general, the more polar and hydrophilic the emulsion and the higher its water content, the more quickly the cations (and EDTA) are released. Details relating to possible coating amounts are explained elsewhere. A very quick release can be disadvantageous, since an excessive accumulation of silver ions in human tissue can cause a cytotoxic effect. Since bacterial cells react more sensitively to silver than animal cells, a moderate release of silver in the range of 50 to 90 μg (conditions as indicated above) is often the best solution.

The release of the antimicrobial composition present in the emulsion may be measured using a simulated wound fluid. In this case, the measured release over the course of 24 h can be 20 to 60 mg per kilogram of emulsion. The 20 to 60 mg here is the sum total of the mass of cations (Ag⁺, Zn²⁺) and EDTA. A suitable measurement method and the composition of the wound fluid are set out in the examples.

The content of the antimicrobial composition in the emulsion may be 0.5% to 5% by weight of the emulsion. Preferably, the emulsion contains 0.5% to 4% by weight, particularly preferably 0.8% to 3.6% by weight of the antimicrobial composition. Furthermore, the emulsion may contain 0.2% to 4.5% by weight or 0.4% to 4% by weight or 0.6% to 3.8% by weight of the antimicrobial composition.

Best of all, the emulsion contains 1.3% to 2.5% by weight of the antimicrobial composition, since in most cases this makes it possible to achieve a very good compromise between product durability, antimicrobial effect and wound healing. This advantage also exists with regard to the following concentration figures:

With respect to the salts of the components of the antimicrobial composition (tetrasodium EDTA, zinc nitrate or zinc sulfate, and silver nitrate), a concentration of 1.6% to 3.7% by weight or better still 1.7% to 3.6% by weight of these components in the emulsion has proven to be particularly advantageous.

With respect to the pure forms of the components of the antimicrobial composition (ionic forms instead of salts), a concentration of 0.8% to 2.1% by weight or better still 0.9% to 2% by weight of these components in the emulsion has proven to be particularly advantageous.

In addition, the components of the antimicrobial composition may be in a certain quantitative ratio with respect to one another. For example, the weight ratio of the weight concentration of EDTA to the sum total of the weight concentrations of Ag⁺ and Zn⁺ in the emulsion may range from 9 to 1 to 9 to 36. Alternatively, the amount-of-substance ratio of the amount of substance of EDTA to the sum total of the amounts of substance of Ag⁺ and Zn⁺ may range from 4 to 1 to 4 to 32. The amount of substance may be determined in the standard unit mol. The ratio of the components to one another is a factor that is involved in determining the size of the proportion of free cations (Ag⁺, Zn⁺) in relation to the cations that are complexed and thus bound to EDTA. It has surprisingly been found that both free cations and free binding sites on the EDTA molecule are advantageous. It is assumed that it is precisely these that are primarily available for the desired reactions against pathogens and biofilms. Preferred ranges derived therefrom are a weight ratio of the weight concentration from 9 to 30 up to 9 to 36 (9 stands for parts of EDTA) and an amount-of-substance ratio of the amount of substance from 4 to 28 up to 4 to 32 (4 stands for parts of EDTA).

The content of polar liquid or of polar liquids in the emulsion may be adapted to the content of the antimicrobial composition in the emulsion. It is recommended to increase the content of polar liquid with increasing concentration of antimicrobial composition. For example, at a concentration of antimicrobial composition of 0.9% to 1.5% by weight, the liquid content is preferably 7% to 11% by weight; at a concentration of antimicrobial composition of 1.6% to 2.2% by weight, it is preferably 12% to 16% by weight; and at a concentration of antimicrobial composition of 2.3% to 2.7% by weight, it is preferably 17% to 20% by weight. This alignment of the concentrations has an advantageous effect on the stability of the emulsion.

The mesh-like substrate may be partially or completely coated with the emulsion. The coating may be on one side or on both sides. If only one side is coated, preferably the first side that is intended for wound contact should be coated. In particular, at least 80% of the area of the first side of the substrate may be coated with the emulsion. Preferably, 90% of the area of the first side of the substrate is coated with the emulsion. It is particularly recommended to coat at least 99% of this area with the emulsion. Openings in the substrate are not taken into account here when determining the area. Typically, the emulsion will be sufficiently viscous to cover these openings. In the case of a rectangular, mesh-like substrate with a size of 6 cm×10 cm, 100% of the substrate would therefore be coated with emulsion if the emulsion covers an area of 60 cm².

After application to a wound, the emulsion is usually distributed in the wound area. In the case of exuding wounds, it is also possible for the emulsion to be liquefied, with the result that generally at least some of the previously coated openings are exposed, which benefits the mass transfer.

An exemplary coating process comprises the following steps:
1) provision of support, roller and emulsion, where the emulsion is present as an emulsion bath,
2) immersion of the roller into the emulsion bath,
3) rotation of the roller in the immersed state in order to completely load it with emulsion,
4) transfer of the emulsion onto the support by rolling the roller over that side of the support which is intended or configured for application to a wound, or by pulling said side of the support over the roller.
   If required, process step 1) may be expanded by additionally heating the emulsion in the emulsion bath (for example to a temperature between 35° C. and 50° C.).

The coating may for example be performed by means of a rotating roller with a surface that first takes up the emulsion and then transfers it to the substrate. The roller may be loaded by immersing it in the emulsion and rotating it in the immersed state. Alternatively, it is possible for the substrate (which is usually present as a mass-produced product in the production process) to be guided via the rotating roller and thus immersed in the emulsion. This normally results in a coating on both sides. Depending on the formulation of the emulsion and the ambient temperature, it may be advantageous to heat the emulsion here. It is possible to spray the emulsion onto the substrate as long as the emulsion is not too viscous. If the viscosity is too high, it is possible to heat the emulsion before the spraying process (for example to 40° C. to 50° C.) in order to reduce the viscosity.

The resulting coating amount may be determined by weighing the treated substrate. Coatings with a relatively great basis weight may be produced by repeated individual coatings.

According to a preferred embodiment of the invention, the wound contact layer comprises a mesh-like substrate with a first and second side that are coated with the emulsion. This offers the advantage that the user does not have to decide which side is configured for application to the wound, which reduces the probability of errors and makes work easier in everyday clinical practice.

The mesh-like substrate of the wound contact layer may contain or consist of at least one of the following materials: polyamide, in particular nylon, polyester, cotton, viscose or a combination of two or more of the materials listed. Said materials may be in the form of fibres. For example, they may be polyamide fibres, in particular nylon fibres, polyester fibres, cotton fibres or viscose fibres. The substrate may here be in the form of a knitted polyamide fabric; in particular, it may be present as a knitted nylon fabric. Furthermore, use may also be made of mixed fibres, which combine a combination of two or more of the materials listed. A substrate in the form of fibres can be loaded with particularly large amounts of emulsion since the emulsion can fill the spaces between fibres.

In an advantageous manner and to achieve particularly pronounced antimicrobial efficacy, the antimicrobial composition may contain 35% to 45% by weight of silver nitrate, 20% to 40% by weight of zinc nitrate and 15% to 30% by weight of tetrasodium EDTA. Alternatively, the components may be present in the antimicrobial composition in the following weight ratio with respect to one another: 1.6 to 2 silver nitrate to 2.9 to 3.3 zinc nitrate to 0.8 to 1.2 tetrasodium EDTA.

The emulsion may contain 0% to 40% by weight of water, preferably 5% to 40% by weight of water or 5% to 30% by weight of water, particularly preferably 10% to 30% by weight of water and very particularly preferably 20% to 30% by weight of water. A higher water content results in a quicker release of the antimicrobial constituents. A lower water content results in a delayed and longer-term release. Emulsions with a lower water content are particularly well suited for preventing an infection. Emulsions with a higher water content are recommended for treating already infected wounds or those wounds that benefit from a higher introduction of moisture, such as wounds with fibrin coatings or necrotic wounds.

The first and optionally also the second side of the mesh-like substrate may be coated with an emulsion amount of 100 to 320 g/m², preferably 110 to 250 g/m², best of all 120 to 200 g/m² (coating amount). It has been found that this amount results in excellent results in the case of promoting wound healing and combatting pathogens in the wound. At the same time, it is possible to obtain a stable product with a sufficiently long shelf life, specifically without the emulsion detaching from the wound contact layer. Preferably, the coating is a uniform or substantially uniform distribution of the emulsion in which each region of the prepared side of the wound contact layer is equipped with the same amount of emulsion.

Alternatively, in the case of a mesh-like substrate coated with emulsion, the ratio of the substrate weight to the emulsion weight may be within the following ranges: 1 to 1.4 to 1 to 2.4, preferably 1 to 1.6 to 1 to 2.2, and particularly preferably 1 to 1.9 to 1 to 2.1.

The mesh-like substrate may for example have a basis weight of 50 to 120 g/m², preferably 70 to 100 g/m² and particularly preferably 80 to 90 g/m². Substrates of this kind are usually thin enough and/or provided with openings such that the transition of fluid through the wound contact layer into a possible secondary dressing is optimized. Furthermore, the emulsion is not significantly retained in such a substrate (in contrast to thicker or denser woven fabrics), with the result that the antimicrobial components and nourishing ingredients are efficiently released to the wound and surrounding skin.

Moreover, the linear density of the mesh-like substrate may be 60 to 100 dtex, preferably 70 to 90 dtex, measured in accordance with DIN EN ISO 2060. The tenacity of the substrate may be 30 to 50 cN/tex, preferably 35 to 40 cN/tex, measured in accordance with DIN EN ISO 2062.

Moreover, the emulsion may comprise at least one constituent selected from the following group: mono-, di- and triglycerides, fatty acid esters, lanolin, an oligomer of glycerol, petroleum jelly, paraffin, synthetic wax or mixtures thereof. The fatty acid esters may be diglycerol esters of mono- and dicarboxylic fatty acids. These constituents make it possible to provide an emulsion that both has atraumatic properties—that is to say can be easily detached from the wound without causing pain—and can absorb excess moisture in the case of exuding wounds or release moisture to dry wounds in the sense of a moist wound treatment. In particular, wax constituents (including lanolin), glycerol derivatives and fatty acid esters can give the emulsion the property of absorbing and releasing moisture.

Preferably, the emulsion comprises a mono-, di- or triglyceride, which is a mono-, di- or triglyceride of at least one of the following acids: caprylic acid, capric acid, isostearic acid, stearic acid or 12-hydroxystearic acid. Glycerides of this kind have particularly good emulsifying properties and can be mixed with the other constituents to form a stable emulsion. Furthermore, these glycerides exhibit excellent shelf life without the tendency towards oxidation or other undesirable reactions.

The emulsion may further contain 3% to 30% by weight of a hydrophilic constituent in addition to a possible water content. Water should thus be disregarded for the specified proportion range. This hydrophilic constituent is a hydrophilic compound and preferably a covalent (molecular) hydrophilic compound. It may be present in its pure form as a liquid or solid (for example powder). Preferably, the emulsion contains 5% to 15% by weight of the additional hydrophilic compound. Alternatively, all polar liquids (i.e. also alcohols such as glycerol in addition to water) may be disregarded for the specified proportion range. Additional hydrophilic compounds include water-binding constituents and/or emulsifiers. One example of a suitable hydrophilic compound is PEG. Admixing this additional hydrophilic compound makes it possible to improve stability, consistency and flow behaviour of the emulsion. In the optimal case, these parameters are adjusted such that the emulsion can be applied as a stable coating of the wound contact layer, but when it comes into contact with a wound the action of moisture and body heat causes it to exhibit creep behaviour, where it passes into the wound and spreads therein.

The additional hydrophilic compound may comprise polymers, alcohols, glycerol or mixtures thereof. Possible mixtures are an alcohol with glycerol or a polymer such as PEG (for example PEG 2000) with an alcohol such as ethanol.

According to one variant, the selected hydrophilic constituent is or contains a polymer which is a polysaccharide or a PEG (for example PEG 2000) or a mixture of these two polymers.

If the selected hydrophilic constituent is a polymer that is a polysaccharide, then this polysaccharide is or comprises cellulose, methyl cellulose, carboxymethyl cellulose, starch, modified starch, alginates, chitosan or mixtures thereof. Examples of modified starch are oxidized starch, monostarch phosphate, distarch phosphate, phosphated distarch phosphate, acetylated distarch phosphate, acetylated starch, hydroxypropyl starch and dextrin. Among these constituents, preference is given to cellulose and the mentioned derivatives of cellulose and chitin, since they cannot be used as a carbon source (nutrient substrate) by most pathogenic microorganisms. Conversely, low molecular weight polysaccharides (for example dextrin) in sufficient concentration can display a strong osmotic potential and thus inhibit the multiplication of microorganisms.

Should the selected hydrophilic constituent be an alcohol, provision may be made for this alcohol to comprise or be ethanol, a sugar alcohol such as glycerol, propylene glycol, ethylene glycol or mixtures thereof.

Furthermore, the selected hydrophilic constituent may be PEG, where PEG for example has a molecular weight of 0.2 to 8 kDa, preferably 1 to 3 kDa, particularly preferably 2 kDa (PEG 2000). Furthermore, PEG may have a maximum molar mass of 600 g/mol, where PEG with such a maximum molar mass is present as a polar liquid. PEG does not have an occlusive effect on the skin or the wound, but instead increases the absorption of secretions into the emulsion and therefore also into the wound contact layer due to the osmotic activity. The lower the molar mass of PEG, the more pronounced this effect is. At the same time, PEG acts as a moisture-binding factor in the emulsion, which is even able to counteract premature drying-out of the emulsion.

The mesh-like substrate may be coated or impregnated with elemental, nonionic silver. In this case, this silver coating exists in addition to the silver content present in the antimicrobial composition. The additional silver coating can further increase the antimicrobial effect, enables a depot effect and can also reduce or even eliminate the ability of pathogens to colonise and multiply in the substrate. The latter is particularly advantageous for infected wounds that emit pathogen-containing exudate, which is absorbed by the wound contact layer.

When determined in accordance with Standard DIN ISO 2176, the dropping point of the emulsion can be above 35° C., preferably between 35° C. and 45° C. and particularly preferably between 35° C. and 37° C. One way to adjust the dropping point is to add specific nonpolar, aliphatic hydrocarbons, oils or fatty acids to the emulsion. In this case, long-chain, saturated compounds increase the dropping point and short-chain or unsaturated compounds decrease the dropping point. A dropping point that is in the approximate range of body temperature offers the advantage that the emulsion spreads better under the action of body heat and thus passes into the wound. The dropping point should however not be selected to be lower than necessary, since this can otherwise adversely affect the stability and storability of the treated wound contact layer.

The wound contact layer can have an extensibility in accordance with DIN EN ISO 1798:2008-04 of at least 25%, preferably of at least 35%. Extensibility should be understood to mean that a corresponding extension of the material under tensile force does not result in a stress fracture or a tearing of fibres. Preferably, the extensibility is reversible extensibility, so that the material essentially assumes its original length after the tensile force has been removed. Reversible extensibility is present even if the material assumes a length of at most 105% of the initial length after the tensile force has been removed. This length has the same spatial orientation as the tensile force, i.e. it must be measured in the direction of the tensile force. Preferably, the specified extension values apply in the fibre direction. A defined fibre direction is given in most textile arrangements.

The release of cations (Ag⁺ and Zn²⁺) from the emulsion with which the substrate is coated is in each case 1 to 200 μg per 100 cm² of coated substrate over the course of 24 h, where the release is effected to a water volume of 100 ml of water, within a period of 24 h and at a temperature of 37° C. Under these conditions, the release is preferably 10 to 180 μg, better still 20 to 160 μg and best of all 30 to 100 μg.

The invention further relates to a wound dressing comprising the wound contact layer according to the invention. The wound contact layer is present here as an outer layer of the wound dressing that is in contact with the skin or the wound during application. Before use, the first side of the wound contact layer may be covered by a protective layer such as a release liner. The protective layer is removed before use.

Preferably, this wound dressing comprises an absorption layer attached to the second side of the wound contact layer and/or a peripheral adhesive edge for adhering the wound dressing to the skin which surrounds the wound and/or a rear-side support layer (backing) (situated above the second side of the wound contact layer). The absorption layer may comprise nonwoven fabric, superabsorbent polymer or a foamed material. The support layer may be formed as a film that is optionally permeable to water vapour, but not to liquid water, and that may contain or consist of a polyurethane (PU). The peripheral adhesive edge may be part of the support layer. An absorption layer is especially suitable for bleeding or highly exuding wounds. A support layer has the advantage that wound and wound contact layer are protected from moisture from the outside, additionally prevents transfer of emulsion to clothing or bed linen and contributes to the preservation of moisture (for example by stabilizing the water content) in the emulsion.

A further aspect of the invention relates to the wound contact layer according to the invention or a wound dressing comprising this wound contact layer for use in a method for treating wounds, preferably infected wounds, particularly preferably infected, biofilm-containing wounds.

A further aspect of the invention relates to an emulsion containing an antimicrobial composition, as described in the present case, for use in a method for treating wounds, preferably infected wounds, particularly preferably infected, biofilm-containing wounds, where the emulsion is present as a coating on a mesh-like substrate which is provided as a wound contact layer.

According to a preferred embodiment of the invention, the support of the wound contact layer is coated with an emulsion containing a water concentration of 20% to 30% by weight in combination with an antimicrobial composition containing more than 15% by weight of EDTA. A concentration of 15% to 92% by weight of EDTA is particularly preferred and a concentration of 15% to 30% by weight is most preferred.

According to another preferred embodiment of the invention, the support of the wound contact layer is coated with an emulsion containing a water concentration of 26% to 29% by weight in combination with a concentration of the antimicrobial composition in the emulsion of 1% to 2% by weight. A concentration of 1.2% to 1.6% by weight of the antimicrobial composition is particularly preferred.

A particularly preferred embodiment relates to a wound contact layer according to the invention wherein the mesh-like substrate is a woven tulle fabric containing polyester or polyamide and wherein the emulsion contains 1.3% to 1.5% by weight of the antibacterial composition, and 26% to 29% by weight of water and also triglycerides and PEG and wherein the antibacterial composition contains 43% to 49% by weight of silver, 24% to 30% by weight of zinc and 24% to 28% by weight of EDTA. The specification “1.3% to 1.5% by weight of the antibacterial composition” may also relate exclusively to the ionic forms of the components of the antibacterial composition.

Furthermore, the features of the two last-mentioned embodiments may be combined with one another. Said features of the two last-mentioned embodiments enable an excellent effect against the dangerous microbe S. aureus, specifically even against the particularly problematic MRSA variants.

In addition, the invention also relates to the use of the wound contact layer for producing the above-described wound dressing or the use of the described emulsion containing the antimicrobial composition in a method for producing the wound contact layer. A possible production method is explained below.

Part of the invention is a method for producing a wound contact layer as described herein, comprising the following steps:
(I) Providing an antimicrobial composition by introducing the following components into a polar liquid:
   a) ionic silver or silver nitrate,
   b) ionic zinc or zinc nitrate or zinc sulfate, and
   c) EDTA.
(II) Producing an emulsion containing the antimicrobial composition provided under (1).
(III) coating at least a portion of the first side of a mesh-like substrate with the emulsion obtained under (II), as a result of which the first side of the mesh-like substrate is configured for application to a wound. The second side may additionally also be coated. Use may likewise be made of a coating process in which both sides are coated simultaneously.

In an optional step (IV), at least the first side of the wound contact layer can be overlaid with a protective layer or protective film, the wound contact layer can be packaged and the packaged wound contact layer can be sterilized.

A further aspect of the invention is a kit containing the wound contact layer according to the invention in combination with a secondary dressing. The secondary dressing should be able to fix the wound contact layer to the wound. To this end, the secondary dressing may for example have adhesive or be wound over the wound contact layer and the relevant body area.

The kit may be a package or a set.

The wound contact layer according to the invention is suitable for the covering and/or therapy of wounds, in particular infected wounds and particularly for the therapy of such wounds having biofilms. For these reasons, the wound dressing may be used in a method for wound therapy and/or for reducing the microbe count in wounds and/or wound biofilms.

### EXAMPLES

### Example 1: Emulsion with Antimicrobial Composition

42 g of antimicrobial emulsion was produced according to the following procedure: First, an aqueous solution of the antimicrobial composition was prepared with the following ingredients:

| | | |
|---|---|---|
| Tetrasodium salt of EDTA: | 800 | mg |
| Silver nitrate: | 1440 | mg |
| Zinc nitrate hexahydrate: | 2480 | mg |
| Water (deionized): | 38,210 | mg |

29,670 mg of this antimicrobial solution was then mixed with the following constituents to form 100 g of emulsion:

| | | |
|---|---|---|
| Triglycerides: | 45,710 | mg |
| Further nonpolar ingredients: | 17,580 | mg |
| PEG 2000: | 7,030 | mg |

As triglycerides, a mixture of saturated C8-Ca fatty acid esters of plant origin (Caprylic/Capric/Stearic Triglyceride) was used. Although this mixture was solid at room temperature, it was miscible with other (nonpolar) substances. It has been shown that body heat causes this mixture to melt, whereupon it obtains outstanding creep properties and spreads excellently without the need for additional measures.

Furthermore, the triglyceride mixture is nontoxic and can be applied to open wounds without problems. On wound edges, the mixture exerts a refatting effect and gives them a hydrophobic coating that counteracts maceration of the wound edges by wound exudate.

Partial esters of diglycerol were used as further nonpolar ingredients. The following compounds were esterified here with diglycerol: fatty acids with medium chain length, isostearic acid, stearic acid, adipic acid and 12-hydroxystearic acid.

Said diglycerol esters can absorb more than 200% by weight of water. It has been found that, after addition of these diglycerol esters, the emulsion has a significantly increased stability at relatively high temperatures. At the same time, the permeability for wound exudate was increased.

A woven tulle fabric made of polyester was selected as the starting material for providing the substrate. The linear density in accordance with DIN EN ISO 2060 was 76 dtex. An area of 20 cm×10 cm was cut out of this material. This cut-out area had a weight of 1.6 g and was subsequently used as substrate for the wound contact layer. The substrate was provided with 2.56 g of the emulsion by means of dip coatings. This corresponded to a coating amount of 128 g/m².

The components of the antimicrobial composition consisted of the salts tetrasodium EDTA, AgNO₃ and Zn(NO₃)₂ and accounted for 3.26% by weight of the emulsion. With respect to the pure components of the antimicrobial composition consisting of EDTA, Ag⁺ and Zn²⁺, this gives a relevant active ingredient content of 1.27% by weight corresponding to 1.27 g of 100 g of emulsion. Of this, 423 mg was EDTA, 494 mg was zinc ions and 356 mg was silver ions. The water content in the emulsion was 27% by weight. The oil content or content of fat-soluble constituents in the emulsion was 63.29% by weight.

### Example 2: Release of the Antimicrobial Composition

To demonstrate the release of the antimicrobial composition from a wound dressing according to the invention coated with emulsion, the release to simulated wound fluid was determined by means of the following scenario:

Provided as substrate in a sufficient quantity were identical woven tulle fabrics a) and b) made of a knitted polyethylene terephthalate fabric each with an area of 3 cm×3 cm and a density of 1.3-1.4 g/cm³. The woven fabrics were coated on one side with emulsion according to the invention, where the emulsion on woven fabric a) contained 1.4% by weight of antimicrobial composition and the emulsion on woven fabric b) contained 1.94% by weight of antimicrobial composition (based on the ionic forms). The antimicrobial composition consisted of 26% by weight of EDTA, 46% by weight of Ag⁺ and 28% by weight of Zn²⁺. The other constituents of the emulsion were analogous to the formulation from Example 1.

10 ml of simulated wound fluid (SWF) was then mixed twice. This consisted to an extent of 50% by volume of bovine serum and to an extent of 50% by volume of aqueous peptone solution. The peptone solution contained the following ingredients per 1 l of culture medium: 3.56 g of potassium hydrogenphosphate, 5.77 g of disodium hydrogenphosphate, 4.3 g of NaCl, 1 g of casein peptone (pancreatic digest).

Woven tulle fabric a) and b) were incubated in 10 ml each of SWF in closed bottles (20 ml volume) at 37° C. on a shaker. Samples were taken after 4 h, 24 h and 72 h. Woven fabric and SWF were kept in motion throughout the entire incubation period so that the emulsion was continuously wetted.

The samples taken were analysed for eluted EDTA in accordance with EN 13368-1 (in each case 4 ml of SWF per time). To this end, the EDTA released into the SWF was complexed with Fe(III) and the resulting complex was detected by HPLC-UV (absorption maximum at 260 nm).

For the analysis of the cations (Ag, Zn) released into the SWF, sample vials were taken from the shaker after the same three incubation times, and the wound dressings were removed from the sample vials. The 10 ml of SWF was admixed with 20 μl of HNO₃ (65% by weight in H₂O) in order to protect the silver ions from undesirable precipitation. The cation concentration was then determined in accordance with DIN EN ISO 11885 by inductively coupled plasma optical emission spectroscopy (ICP-OES).

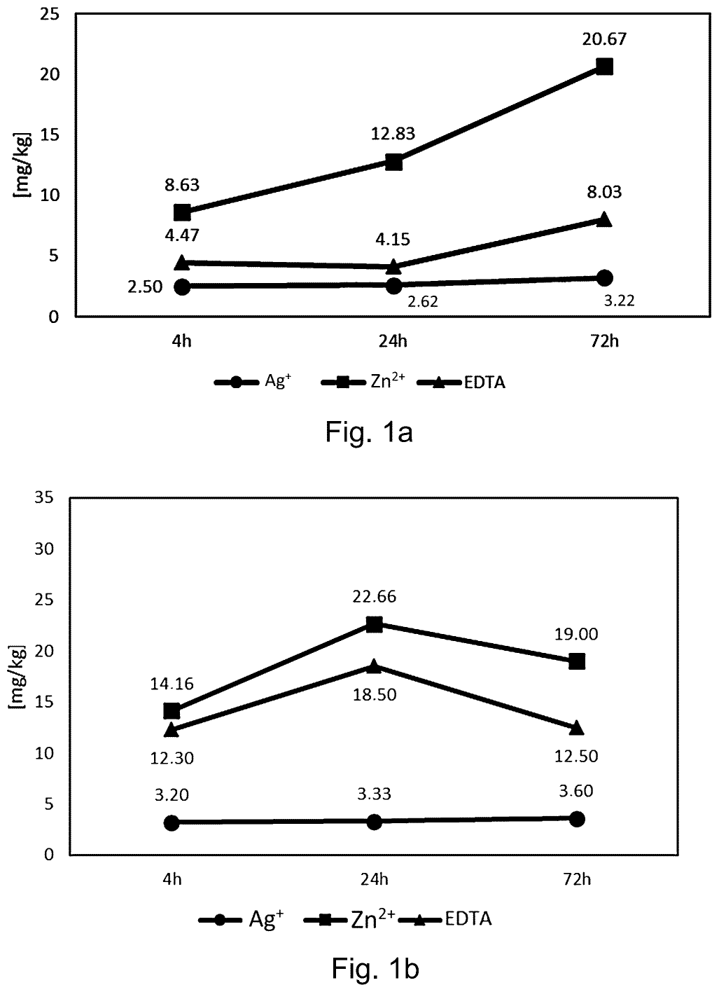

The results are shown in FIG. 1a (1.4% of antimicrobial composition in the emulsion) and FIG. 1b (1.94% of antimicrobial composition in the emulsion).

### Example 3: Demonstration of the Efficacy of Wound Contact Layers According to the Invention Against Biofilms by Means of a CDC Biofilm Reactor®

The test was carried out using a test system, namely the CDC Biofilm Reactor® (available from BioSurface Technologies Corporation, USA). The CDC Biofilm Reactor® is referred to as “reactor” below. The aim of the test was the production of a biofilm and the measurement of the antimicrobial efficacy of wound contact layers according to the invention against such a biofilm. A test of this kind can be used to simulate the efficacy of substances which are intended to combat a biofilm in wounds.

The interior of the reactor used had eight rods, each of which had three samples attached to it. The reactor vessel therefore contained twenty-four samples. These samples were mounted in the form of what are known as coupons. The coupons were round supports with surfaces that enabled microorganisms to adhere and combine to form a biofilm.

During the experimental procedure, nutrients for cell proliferation were introduced into the reactor continuously and in a constant amount and discharged again so that a constant volume prevailed.

The protocol used was a slightly modified version of Standard ASTM E2871—13:

Overnight cultures of the two bacterial species S. aureus (strain ATCC 6538) and P. aeruginosa (strain ATCC 15442) were prepared. To this end, in each case 10 ml of TSB medium (tryptic soy broth) was inoculated with a single colony and incubated at 37° C. on an orbital shaker.

The overnight culture of P. aeruginosa was adjusted to 1×10⁸ CFU/ml (colony forming units). 1 ml of this culture was then used to inoculate 300 ml of TSB. This medium was transferred into the reactor and further cultivation was carried out in this reactor in batch phase, specifically at 37° C. and 80 rpm on a magnetic stir plate.

After 24 hours of biofilm growth, the rods were washed twice with PBS medium (phosphate-buffered saline) and the now biofilm-containing coupons were transferred into 12-well plates. Each coupon was wrapped here with samples of the wound contact layer according to the invention (2.5 cm×5 cm). The subsequent incubation lasted 24 h and was carried out at room temperature (P. aeruginosa) or 37° C. (S. aureus). The coupons were then removed from the well recesses, transferred into 10 ml of Dey-Engley neutralizing broth and subjected to ultrasound treatment for 30 min.

The samples were briefly shaken by means of a vortex device and distributed on 96-well plates. This was followed by a serial dilution of each sample (1:10 in PBS), which was then plated out on TSA. 20 μl per dilution was used here (duplicate preparation for each dilution of each sample). The plates were incubated at 37° C. overnight. The colonies were counted the following day.

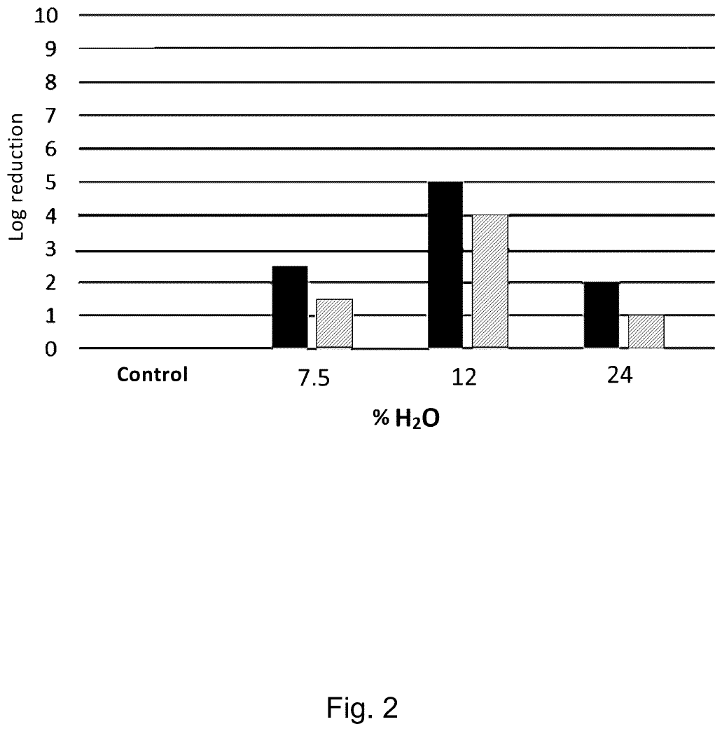

The results are shown in FIG. 2 for wound contact layers with a concentration of antimicrobial composition of 0.94% by weight (based on the ionic forms).

### Example 4: Demonstration of the Efficacy of Wound Contact Layers According to the Invention Against Biofilms by Means of a Drip Flow Bioreactor

Further tests were carried out on biofilms produced in a drip flow reactor. Analysis by means of drip flow reactor takes place with ingress of air and is more similar to the conditions prevailing in a wound. These tests took place in accordance with Standard ASTM E2647—13, however with minimal modifications in order to generate data from various microorganism species. The organisms used can be found in the following table:

TABLE I

| | | Deposit No. |
|---|---|---|
| | | or strain |
| Gram-positive organisms | Staphylococcus aureus | ATCC 29213, |
| | | MRSA ATCC |
| | | BAA-43 |
| | Staphylococcus epidermidis | ATCC 35984 |
| | Enterococcus faecalis | ATCC 29212 |
| Gram-negative organisms | Pseudomonas aeruginosa | ATCC 15442 |
| | | ATCC 700888 |
| | Acinetobacter baumannii | ATCC 19606 |
| | Klebsiella pneumoniae | ATCC 700603 |
| Fungal organisms | Candida albicans | ATCC 10231 |
| | Candida auris | NCPF 8971 |

First, 10 ml of TSB was inoculated with a single colony of each strain and prepared as an overnight culture at 37° C. and 125 rpm on a shaker incubator.

Absorbent pads were attached to the slides by means of adhesive and the chambers of the drip flow reactor were loaded with the slides. The reactor was then sterilized in an autoclave.

The absorbent pads were moistened with 1 ml of TSB and equipped with polycarbonate filters (2 cm×2 cm; 0.2 μm). The overnight cultures were adjusted to 1×10⁸ CFU/ml. The filters were then wetted with 10 μl of this solution and left to dry.

The covers of the reactor were attached and the reactor was attached to a 10 l carboy (270 mg/l of TSB). The hose for the nutrient solution was connected to a pump and the nutrients were supplied at a rate of 5 ml/min/chamber.

After 24 h, the wound contact layers according to the invention were cut to a size of 2.5 cm×2.5 cm and completely saturated with simulated wound fluid (50:50—Maximum Recovery Diluent:fetal bovine serum). The wound contact layers were then placed on the top of the inoculated filters, the covers of the chambers were secured and the reactor was put into operation again for 24 h.

After the time had elapsed, the filters were removed, placed in 10 ml of Dey-Engley neutralizing broth and sonicated for 30 min. The samples were distributed on 96-well plates and a 1:10 dilution series was generated from each sample with PBS. The dilutions were plated out on TSA (20 μl). The colonies were counted after incubation at 37° C. overnight.

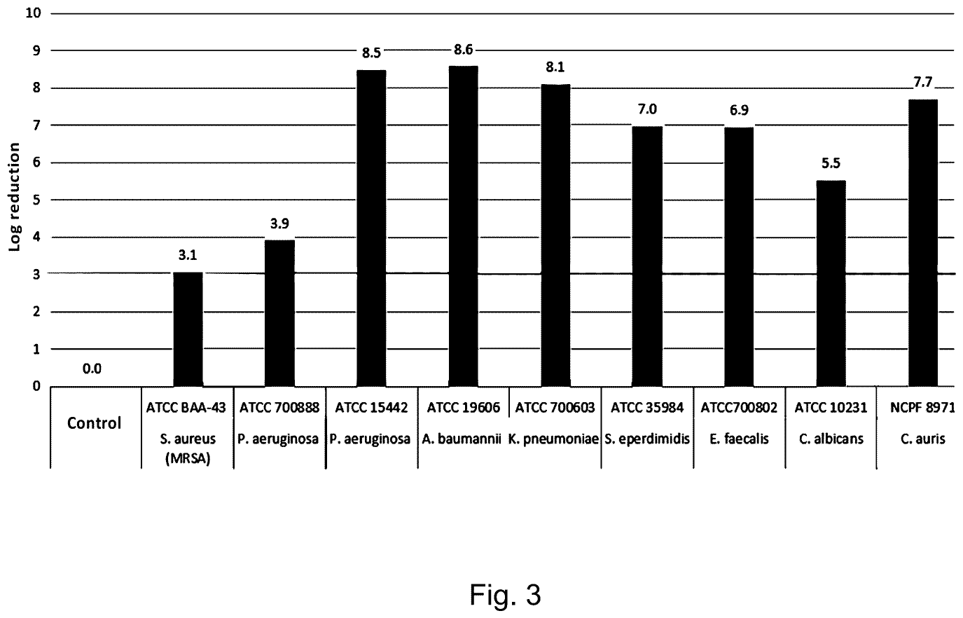

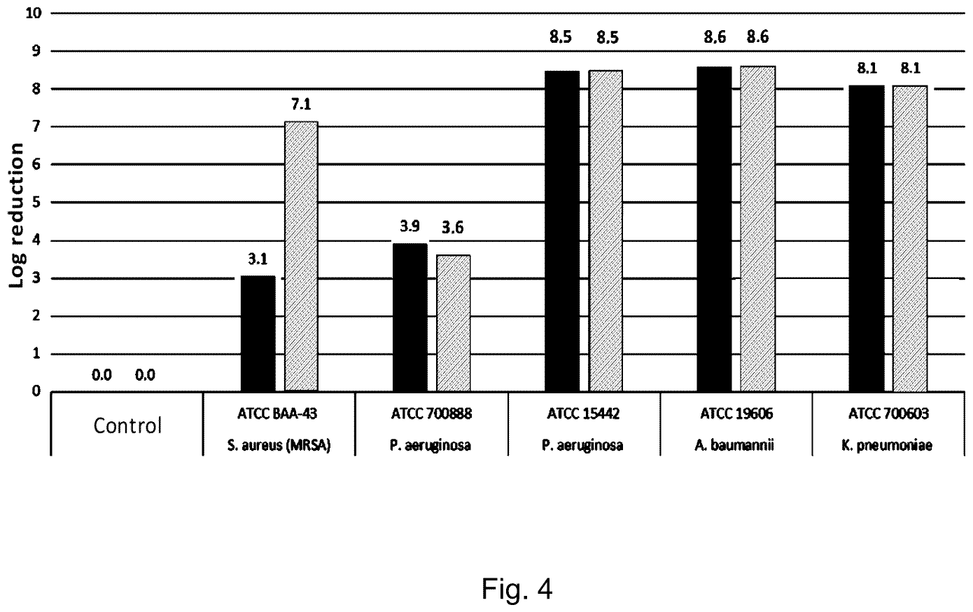

The results of the experimental evaluation are shown as logarithmic reduction in the cell count of the tested organisms in FIG. 3 and FIG. 4. As can be seen from FIG. 4, the efficacy against methicillin-resistant Staphylococcus aureus (MRSA) was able to be significantly increased by increasing the EDTA content.

### Example 5: Demonstration of Antimicrobial Efficacy in a Wound Contact Layer Test in Accordance with AATCC 100

The test was used to demonstrate the efficacy of the antimicrobial effect of wound dressings according to the invention in a contact test and was performed in accordance with Standard AATCC 100 according to the following scheme:

An overnight culture of the respective microorganism was prepared by inoculation of 10 ml of TSB with a single colony. The subsequent incubation took place at 37° C. and 125 rpm on a shaker incubator. The overnight cultures were adjusted to 1×10⁸ CFU/ml.

Wound contact layers according to the invention which contained an emulsion with 1.4% by weight of antimicrobial composition (based on the ionic forms) were used. The antimicrobial composition consisted of 26% by weight of EDTA, 46% by weight of Ag⁺ and 28% by weight of Zn²⁺. The wound contact layers were each tested in triplicate. To this end, the wound contact layers were transferred into Petri dishes (one contact layer per Petri dish) and wetted with 0.85% NaCl solution. The wound contact layers were then individually introduced into 50 ml Falcon tubes, admixed with 10 ml of TSB and inoculated with 100 μl of the overnight culture so that the target concentration was 1×10⁶ CFU/ml.

The wound contact layers were incubated at 37° C. and 125 rpm on a shaker incubator. 1 ml of each sample was then transferred into new 50 ml Falcon tubes and 9 ml of neutralization solution was added.

The following step was performed in a 96-well plate, where 200 μl of cell solution was filled into the wells of the first plate column. 180 μl was added to the wells of plate columns 2 to 7. A 10-fold serial dilution of the cell solution was then prepared by transferring 20 μl of the solution into the respective following well columns.

From each dilution, 50 μl was plated out in duplicate on TSA agar plates and incubated at 37° C. overnight. The colonies were counted the following day.

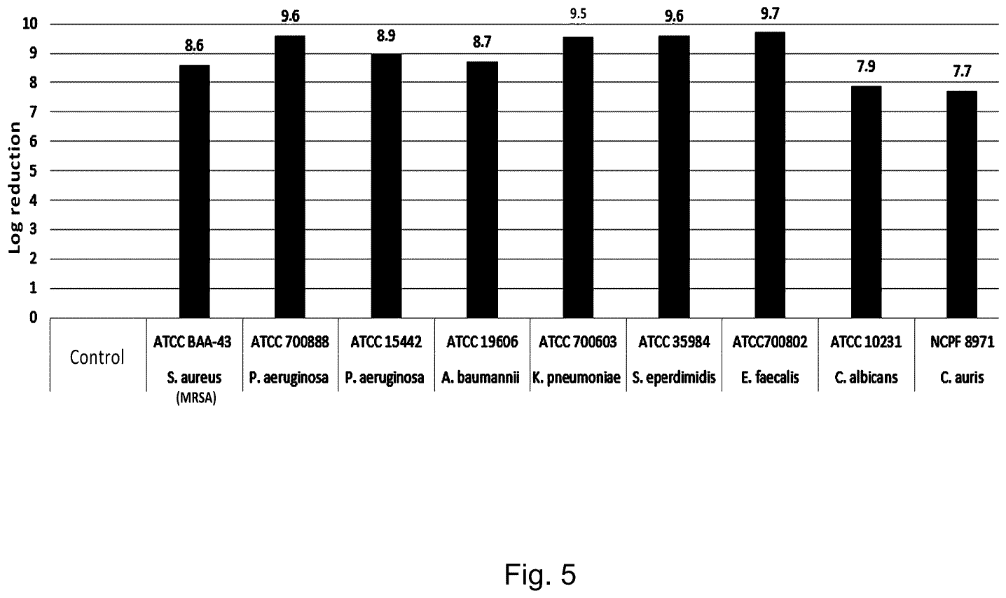

The results are shown in FIG. 5 as average values.

### Example 6: Determination of Viscosity and Dropping Point

Three emulsions according to the invention were provided, where each emulsion contained a different concentration of antimicrobial composition. Emulsions with concentrations of respectively 0.94%, 1.4% and 1.94% of antimicrobial composition (based on the ionic forms) were selected for the test.

To determine the dropping point, the respective samples were filled at room temperature into the heating block of a suitable measuring device and this was heated at a rate of 1° C./min. As soon as the first droplet of the molten sample fell through an opening in the bottom of the heating block and thus passed a light barrier, the sample temperature was recorded. This procedure was performed a total of three times for each emulsion and the values were then averaged.

The resulting dropping points can be found in the following table:

TABLE II

| | | | |
|---|---|---|---|
| Concentration of the | 0.94 | 1.4 | 1.94 |
| antimicrobial composition [%] | | | |
| Dropping point of the | 38.3 | 38.6 | 39.3 |
| emulsion [° C.] | | | |

All of the determined dropping points are close to body temperature. The action of body heat increases the flow property of the emulsion, which increases its release to the wound. The dropping point is not however reached here, and so the emulsion remains stable.

### Example 7: Substrates for Wound Contact Layers

Provided were the following substrates, which are suitable for use as wound contact layers and can be coated with the emulsion according to the invention:

### 1) Substrate Made of Polyamide:

| | |
|---|---|
| Fibre arrangement: | Knitted fabric in the form of a tulle |
| Material: | Nylon 6.6 |
| Thickness: | 0.22 mm |
| Area: | 10 cm × 10 cm |

### 2) Substrate Made of Polyethylene Terephthalate:

| | | |
|---|---|---|
| Fibre arrangement: | Knitted fabric | |
| Linear density: | 76 dtex | (DIN EN ISO 2060) |
| Elongation at break: | 26% | (DIN EN ISO 2062) |
| Strength: | 37 cN/tex | (DIN EN ISO 2062) |
| Density: | 1.3-1.4 g/cm³ | |

3) Substrate with Elemental, Nonionic Silver in the Fibre Structure:

| | |
|---|---|
| Fibre arrangement: | Knitted fabric in the form of a tulle |
| Material: | 90% polyamide in the form of nylon 6.6 |
| | and 10% elemental silver |
| Thickness: | 0.22 mm |
| Area: | 10 cm × 10 cm |
| Basis weight: | 33 g/m² |

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms. The use of the terms “a” and “an” and “the” and “at least one” and similar referents in the context of describing the invention (especially in the context of the following claims) are to be construed to cover both the singular and the plural, unless otherwise indicated herein or clearly contradicted by context.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

1. A wound contact layer comprising a mesh-like substrate which is partially or completely coated with an emulsion, wherein the emulsion comprises an antimicrobial composition containing:
a) ionic silver and/or silver nitrate,
b) ionic zinc and/or zinc nitrate and/or zinc sulfate, and
c) EDTA and/or tetrasodium EDTA.

2. The wound contact layer according to claim 1, wherein the mesh-like substrate comprises at least one of the following fibres: polyamide fibres, polyester fibres, cotton fibres, or viscose fibres.

3. The wound contact layer according to claim 2, wherein the mesh-like substrate comprises nylon fibres.

4. The wound contact layer according to claim 1, wherein the mesh-like substrate consists of at least one of the following fibres: polyamide fibres, polyester fibres, cotton fibres, viscose fibres, or combinations thereof.

5. The wound contact layer according to claim 1, wherein the mesh-like substrate is in the form of knitted fabric or tulle.

6. The wound contact layer according to claim 1, wherein the antimicrobial composition
a) comprises 5% to 50% by weight of one of the following components: ionic silver, or silver nitrate, and
b) comprises 3% to 45% by weight of one of the following components: ionic zinc or zinc nitrate or zinc sulfate, and
c) comprises 15% to 90% by weight of EDTA or tetrasodium EDTA.

7. The wound contact layer according to claim 1, wherein the antimicrobial composition comprises 15% to 30% by weight of tetrasodium EDTA, 35% to 45% by weight of silver nitrate, and 20% to 40% by weight of zinc nitrate.

8. The wound contact layer according to claim 1, wherein the emulsion comprises 5% to 40% by weight of water.

9. The wound contact layer according to claim 1, wherein the emulsion comprises 0.5% to 5% by weight of the antimicrobial composition.

10. The wound contact layer according to claim 1, wherein the first and optionally also the second side of the mesh-like substrate is coated with an emulsion amount of 100 to 320 g/m².

11. The wound contact layer according to claim 10, wherein the second side of the mesh-like substrate is coated with a second emulsion amount of 100 to 320 g/m².

12. The wound contact layer according to claim 1, wherein the emulsion comprises mono-, di- and triglycerides, fatty acid esters, lanolin, an oligomer of glycerol, petroleum jelly, paraffin, synthetic wax, or mixtures thereof.

13. The wound contact layer according to claim 12 wherein the emulsion comprises a mono-, di- or triglyceride which is a mono-, di- or triglyceride of caprylic acid, capric acid, isostearic acid, stearic acid or 12-hydroxystearic acid.

14. The wound contact layer according to claim 1, wherein the emulsion additionally comprises 3% to 30% by weight of a further hydrophilic constituent in addition to a possible water content.

15. The wound contact layer according to claim 14, wherein the hydrophilic constituent comprises polymers, alcohols, glycerol, or mixtures thereof.

16. The wound contact layer according to claim 14, wherein the hydrophilic constituent is a polymer and wherein the polymer is either a polysaccharide or polyethylene glycol (PEG).

17. The wound contact layer according to claim 16, wherein the polymer is a polysaccharide and wherein the polysaccharide comprises cellulose, starch, modified starch, alginates, chitosan, or mixtures thereof.

18. The wound contact layer according to claim 14, wherein the hydrophilic constituent is an alcohol and wherein the alcohol comprises ethanol, glycerol, propylene glycol, ethylene glycol, or mixtures thereof.

19. The wound contact layer according to claim 1, wherein the mesh-like substrate is a woven tulle fabric comprising polyester or polyamide, wherein the emulsion comprises 1.3% to 1.5% by weight of the antibacterial composition, comprises 26% to 29% by weight of water, and comprises triglycerides and PEG, and
wherein the antibacterial composition contains 43% to 49% by weight of silver, 24% to 30% by weight of zinc and 24% to 28% by weight of EDTA.
